(19) United States
(12) Patent Application Publication
(10) Pub. No.: US 2010/0083427 A1
(43) Pub. Date: Apr. 8, 2010

(54) DEVICE FOR ATTACHING A DESIGN ELEMENT TO SWIM GOGGLES

(76) Inventor: MEGAN SCHNURR, Toronto (CA)

Correspondence Address: KELLEY DRYE & WARREN LLP, 3050 K STREET, NW, SUITE 400, WASHINGTON, DC, 20007, US

(21) Appl. No.: 12/244,332

(22) Filed: Oct. 2, 2008

(51) Int. Cl. A61F 9/02; A41D 27/08

(52) U.S. Cl. 2/452; 2/244

(57) ABSTRACT

A device for attaching a design element to a strap of a swim goggle is disclosed. The design element is attached to a substantially planar member, which is secured to the swim goggle strap through a connecting member. The substantially planar member and the connecting member may have various shapes, sizes and orientations. The substantially planar member and the connecting member may be made of soft, flexible material, which is not capable of slicing or cutting the swim goggle strap. The substantially planar member and the connecting member may be constructed without any sharp edges to prevent injury to the swimmer. The substantially planar member and the connecting member may be made of non-corroding material, and which is not subject to discoloration due to the presence of chlorine and other chemicals in swimming pool water.

### FIELD OF THE INVENTION

The present invention relates to a device for attaching a design element to a strap of a swim goggle and other sports or protective eye ware.

### BACKGROUND OF THE INVENTION

There is a need to identify and recognize swimmers, especially swimmers taking part in competitive swimming. These swimmers, with a little more than a portion of their head visible above water are difficult to identify and recognize.

Over the years swim goggles have been transformed into an essential tool for swimmers, be it a recreational swimmer splashing around in a pool or a competitive swimmer at an Olympic event. In addition to greatly improving a swimmer's vision, the swim goggle protects the swimmer's eyes from the chemicals and potentially harmful micro-organisms and bacteria found in many swimming pools. Indeed, it is hard to visualize a swimmer, especially competitive ones, swimming without having a swim goggle strapped to their head.

The present invention discloses a device for attaching a design element to a swim goggle to enable the swimmers to be easily identified and recognized. In addition to functioning as an identification device, the design element may serve various other purposes, including, but not limited to, display of ornaments, display of sponsor's logo, and display of trademarks. Identification and ornamentation devices for use with eye wear have previously been disclosed in the patents to McNulty U.S. Pat. No. 5,497,211 and Mendola U.S. Pat. No. 4,968,128. The eyeglass frame ornamentation of the Mendola '128 patent describes an attachment for the removable placement of conventional ornaments such as ear rings, pins, medals, charms to the frames of eyeglasses. The use of a rigid strap-like clamp taught by the Mendola '128 patent may not be suitable for use with swim goggles as the rigid strap-like clamp will likely damage or tear the swim goggle strap. The indicia display device of the McNulty '211 patent discloses a display pennant, an elongated support and a fastener for coupling the device with an eyeglass temple-piece. It would be difficult if not impossible to use the display device disclosed by the McNulty '211 patent with swim goggles because the display pennant and the elongated support would not be aerodynamic and would create additional drag for the swimmer. In addition, the elongated support would make the display device susceptible to frequent damages due to the forces encountered by the display pennant and the elongated support when the swimmer dives into the water and swims.

### SUMMARY OF THE INVENTION

Responsive to the foregoing challenges, Applicant has developed an innovative device for attaching a design element to a strap of a swim goggle, comprising: a substantially planar member, the substantially planar member having a first side and a second side, the first side having design element therein; and a connecting member adapted to secure the substantially planar member to a swim goggle strap.

Applicant has further developed an innovative device for attaching a design element to a strap of a swim goggle, said device comprising: means for displaying a visually perceptible design element having a shape adapted to minimize tangle with a swim goggle wearer's hair; and means for connecting the displaying means to the strap of the swim goggle.

Applicant has still further developed an innovative device for removably attaching a design element to a strap of a swim goggle, the device being adapted to minimize hair tangle and constructed of soft, flexible material without any sharp edges, said device comprising: a substantially planar member made of non-corroding material and not subject to discoloration due to chlorinated water, the substantially planar member having a first side and a second side, the first side having a design element therein, the design element being readily removable and changeable; and a connecting member adapted to secure the substantially planar member to a swim goggle strap.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE DRAWINGS

In order to assist the understanding of this invention, reference will now be made to the appended drawings, in which like reference characters refer to like elements.

FIG. 1 is a first side view of an exemplary embodiment of a device for attaching a design element to swim goggles.

FIG. 2 is a second side view of an exemplary device of FIG. 1.

FIG. 3 is a cross sectional view of an exemplary device of FIG. 1.

FIG. 4 is a cross sectional view of an exemplary embodiment of a device for attaching a design element to swim goggles.

FIG. 5 is a cross sectional view of an exemplary embodiment of a device for attaching a design element to swim goggles.

FIG. 6 is a first side view of an exemplary device of FIG. 4.

FIG. 7 is a second side view of an exemplary device of FIG. 4.

FIG. 8 is a side view of an exemplary embodiment of a device for attaching a design element to swim goggles.

FIG. 9 is a cross sectional view of an exemplary device of FIG. 8.

### DETAILED DESCRIPTION OF EMBODIMENTS

The device for attaching the design element to the strap of a swim goggle may be comfortable to the wearer and may be designed such that its position can be adjusted along the strap of the goggle. The device may be made of material which is soft, flexible and unable to slice or cut the goggle strap. The device may not have any sharp edges so that injury or cuts to the swimmer is prevented.

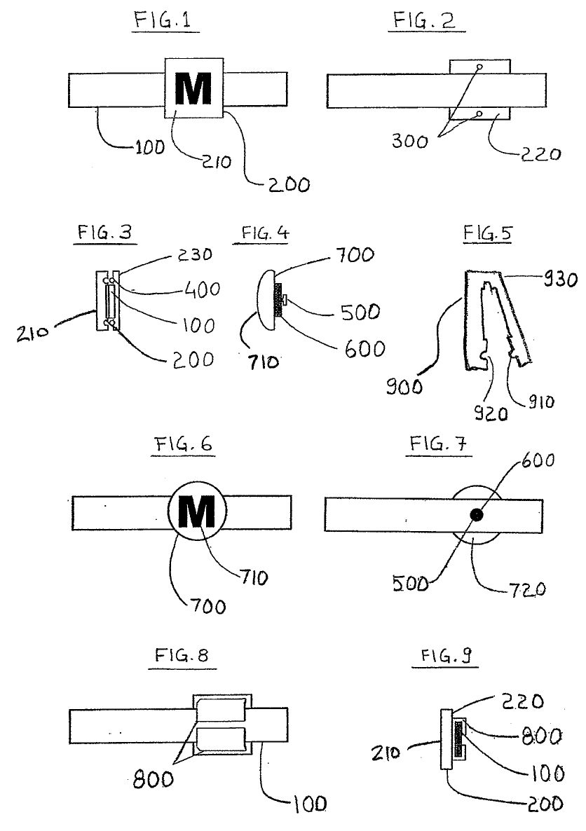

Reference will now be made in detail to a first embodiment of the present invention, an example of which is illustrated in the accompanying drawings. FIGS. 1, 2 and 3 show an embodiment of the device for attaching a design element to a strap of a swim goggle. FIG. 3 is a cross-sectional end-on view of the exemplary embodiment, while FIGS. 1 and 2 are side views of the exemplary embodiment of a device for attaching a design element to a swim goggle strap 100. FIG. 1 shows a substantially planar member 200, the substantially planar member having a first side 210 and a second side 220. As shown in FIG. 1, the first side 210 of the substantially planar member may be adapted to receive and hold in place, incorporate, or constitute itself, a design element. As shown in FIG. 2, the second side of the substantially planar member 220 may have a pair of apertures 300, the apertures 300 being spaced apart in a predetermined manner. As shown in FIG. 3, these apertures may be adapted to matingly engage with male connectors 400. The male connectors 400 are disposed on and extend outwards from a second substantially planar member 230. As shown in FIG. 3, the substantially planar member 200 containing the design element may be attached to the strap of the swim goggle 100 by placing its second side 220 adjacent to and facing the swim goggle strap. The male connectors 400 may then be pushed from the other side of the swim goggle strap through the aperture 300 to removably attach the substantially planar member 200 to the strap of the swim goggle.

FIGS. 4, 6 and 7 show another embodiment of the device for attaching a design element to a strap of a swim goggle. FIGS. 4 is a cross-sectional end-on view of the exemplary embodiment, while FIGS. 6 and 7 are side views of the exemplary embodiment of the device for attaching a design element to a swim goggle strap 100 shown in FIG. 4. FIG. 4 shows a member 700 with a substantially planar back or second side 720 and a rounded or convex front or first side 710. As shown in FIG. 6, the first side 710 of the substantially planar member may be adapted to receive and hold in place, incorporate, or constitute itself, a design element. The second side of the substantially planar member 720 may have a centrally located aperture 300. The aperture 300 may be adapted to receive a body of a snap-fastener 500. As shown in FIG. 4, the snap-fastener 500 may have a head and a body, the body extending outward from the head and adapted to matingly engage the member 700 when the body is inserted within the aperture. As shown in FIG. 4, the member 700 containing the design element may be removably attached to the strap of the swim goggle 100 by placing its second side 720 adjacent to and facing the swim goggle strap. The snap-fastener 500 is then pushed from the other side of the swim goggle strap, through the swim goggle strap and into the aperture 300 such that the body of the snap fastener 500 matingly engage the substantially planar member 200. The swim goggle strap may have holes or an aperture through which the swim goggle snap-fastener 500 is inserted into the aperture 300 of the substantially planar member 700. The snap fastener 500 may fit snuggly against the goggle strap to reduce the likelihood that hair tangles in and/or around the snap fastener.

FIG. 5 shows another embodiment of the device for attaching a design element to a strap of a swim goggle. FIG. 5 is a cross-sectional end-on view of the exemplary embodiment of a device for attaching a design element to a swim goggle strap 100 (shown in other figures). FIG. 5shows a device 900, wherein one surface of the device is adapted to receive and hold in place, incorporate, or constitute itself, a design element. The device 900 may include prong fasteners, where one end of the prongs 930 are hinged to each other, preferably without any exposed hinge elements. The hinge attachment may simply be formed by utilizing flexible/bendable material for the device 900. The other ends of the prongs may have an aperture 920 and a ball shaped or other male projection 930, respectively. The aperture 920 and the ball shaped projection 930 matingly engage around the strap of the swim goggle to removably attach the device 900 for attaching a design element to the strap of a swim goggle.

FIGS. 1, 8 and 9 illustrate yet another embodiment of the device for attaching a design element to a strap 100 of a swim goggle. FIGS. 1 and 8 are side views of the exemplary embodiment, while FIG. 9 is a cross-sectional end-on view of the exemplary embodiment of a device for attaching a design element to a swim goggle strap 100. FIG. 1 shows a substantially planar member 200, the substantially planar member having a first side 210 and a second side 220. As shown in FIG. 1, the first side 210 of the substantially planar member may be adapted to receive and hold in place, incorporate, or constitute itself, a design element. As shown in FIG. 9, a fastener 800 may be attached to the second side 220 of the substantially planar member for securing the device to the swim goggle strap. As shown in FIGS. 8 and 9, the fastener 800 may have a central portion and a first and a second arms extending outward from the central portion, the arms being bent towards each other. The fastener 800 may be any kind of rigid body, such as a C-shaped bracket and may have various shapes, sizes and orientations. As shown in FIGS. 8 and 9, the fastener slidably attaches the device to a swim goggle strap when the strap is pushed through the fastener's bent arms.

With regard to all embodiments, the members 200, 230, 700 and 900 may have various shapes, sizes and orientations without departing from the intended scope of the present invention. The members 200, 230, 700 and 900 may be made of soft, flexible material, which is not capable of slicing or cutting the swim goggle strap. The substantially planar members 200, 230 and 700 may be constructed without any sharp edges to prevent injury to or discomfort for the swimmer or damage to the goggle strap. The members 200, 230, 700, 800 and 900 may be constructed so that they are easily removable from the swim goggle strap. The members 200, 230, 700, 800 and 900 may include few if any exposed elements so as to reduce the likelihood of tangling with a goggle wearer's hair. Further, the members 200, 230, 700, 800 and 900 may be made of material, which does not rust in water, or discolor easily in chlorinated water.

The design element may be placed in or on the first side 210 and 710 of the members 200 and 700 shown in FIGS. 4 and 9, or on either side of the members 200 and 900 shown in FIGS. 3 and 5. The design element may have various shapes, sizes and orientations. The design element may be adapted to be easily removable and/or changeable, may be integrated into the structure of the device or added on by an adhesive. The design element may be made of non-corroding material, which does not rust in water. The design element may be made of material which is not subject to discoloration due to the presence of chlorine and other chemicals in swimming pool water.

It will be apparent to those skilled in the art that variations and modifications of the present invention can be made without departing from the scope or spirit of the invention. For example, size shape and materials may be changed without departing from the intended scope of the invention and appended claims. It is further appreciated that forming one or more elements of the apparatus embodiments of the present invention integrally as opposed to separately is intended to fall within the scope of the invention and appended claims.

What is claimed is:

1. A device for attaching a design element to a strap of a swim goggle, said device comprising:
a substantially planar member, the substantially planar member having a first side and a second side, the first side having design element therein; and
a connecting member adapted to secure the substantially planar member snuggly to a swim goggle strap.

2. The device of claim 1, wherein the second side has a pair of apertures, the apertures being spaced apart in a predetermined orientation; and
the connecting member having a pair of male connectors disposed on a side of the connecting member, the male connectors extending away from the side of the connecting member and spaced apart in predetermined orientation.

3. The device of claim 2, wherein each of the male connectors are adapted to matingly engage the apertures provided in the second side of the substantially planar member.

4. The device of claim 3, wherein the substantially planar and the connecting members are made of non-corroding material.

5. The device of claim 4, wherein the substantially planar and the connecting members are made of materials which do not discolor when immersed in swimming pools containing chlorine and other chemicals.

6. The device of claim 3, wherein the substantially planar and the connecting members are made of soft, flexible material, which is not capable of readily cutting or slicing the goggle strap.

7. The device of claim 6, wherein the substantially planar and the connecting members are constructed without any sharp edges.

8. The device of claim 1, wherein the second side of the substantially planar member has an aperture, and the device further comprises:
a snap-fastener having a head and a body, the body extending outward from the head and adapted to matingly engage the substantially planar member when the body is inserted within the aperture.

9. The device of claim 8, wherein the aperture is centrally located in the second side of the substantially planar member.

10. The device of claim 1, wherein the connecting member is a fastener adapted to releasably attach the substantially planar member to the swim goggle strap.

11. The device of claim 10, wherein the fastener is attached to the second side of the substantially planar member.

12. The device of claim 11, wherein the fastener has a central portion and first and second arms extending outward from the central portion, the arms being bent towards each other.

13. A device for attaching a design element to a strap of a swim goggle, said device comprising:
means for displaying a visually perceptible design element having a shape adapted to minimize tangling with a swim goggle wearer's hair; and
means for connecting the displaying means to the strap of the swim goggle.

14. The device of claim 12, wherein the display means and the connecting means are fixedly connected at one end; and
the other end of the display means and the connecting means are adapted to matingly engage around the strap of the swim goggle.

15. The device of claim 13, wherein the display means and the connecting means are made of non-corroding material.

16. The device of claim 15, wherein the display means and the connecting means are made of materials which do not discolor when immersed in swimming pools containing chlorine and other chemicals.

17. The device of claim 16, wherein the display means and the connecting means are made of soft, flexible material, which is not capable of readily cutting or slicing the goggle strap.

18. The device of claim 17, wherein the display means and the connecting means are constructed without any sharp edges.

19. A device for removably attaching a design element to a strap of a swim goggle, the device being adapted to minimize hair tangle and constructed of soft, flexible material without any sharp edges, said device comprising:
a substantially planar member made of non-corroding material and not subject to discoloration due to chlorinated water, the substantially planar member having a first side and a second side, the first side having a design element therein, the design element being readily removable and changeable; and
a connecting member adapted to secure the substantially planar member to a swim goggle strap.
